(19) United States
(12) Patent Application Publication
(10) Pub. No.: US 2002/0106681 A1
(43) Pub. Date: Aug. 8, 2002

(54) High definition electrical impedance tomography methods for the detection and diagnosis of early stages of breast cancer

(76) Inventors: Alvin Wexler, Winnipeg (CA); Rajen Manicon Murugan, Nashua, NH (US)

Correspondence Address: Shapiro Cohen, Station D, P.O. Box 3440, Ottawa, ON, K1P 6P1, CA

(21) Appl. No.: 09/991,993

(22) Filed: Nov. 26, 2001

Related U.S. Application Data
(60) Provisional application No. 60/252,591, filed on Nov. 24, 2000.

(51) Int. Cl. C12Q 1/68; G06F 19/00; G01N 33/48; G01N 33/50; G01F 1/64; G01N 27/26

(52) U.S. Cl. 435/6; 205/777.5; 702/19; 702/25

(57) ABSTRACT

The HDEIT method of the present invention permits one to use a variety of such indices to distinguish a tumour from normal surrounding tissue because it produces the value of the tissue characteristic at each zone in the tissues measured in accordance with the applied frequency. The tumour distinguishing analysis may be applied to the HDEIT image, or may be applied to the data that comprise the image without generating the image. Such methods are intended to permit the detection of tumors that are too small to be accurately seen in an image, but produce a large enough index for diagnostic purposes. One can apply this capability of the HDEIT method in a number of ways. For example, one can quickly scan the breast at low resolution, perform a distinguishing analysis for tumors, and then only perform a longer-duration high resolution scan if there was an indication of a diagnostically significant area to be examined.

This application relates to U.S. provisional application No. 60/252,591 filed Nov. 24, 2000.

### FIELD OF INVENTION

This invention relates to improved methods for High Definition Electrical Impedance Tomography (HDEIT) for imaging within a region (e.g. human body organs, geophysical structures) which region has inhomogeneous and contrasting electrical conductivity (including dielectric constant, magnetic permeability and, more generally, specific impedance) with specific application to the detection and diagnosis of early stages of breast cancer.

### BACKGROUND TO THE INVENTION

There is a constant effort being made to find improved methods of detecting malignant tumors of the breast. X-ray technology is at present the generally accepted procedure, although it has serious limitations. Frequently patients experience severe discomfort and pain due to the distortion of the breast for irradiation, and the results of the X-ray require expert interpretation, and frequently repetition, followed by a biopsy to confirm the character of a suspected tumor.

Early research into the use of electrical impedance tomography for location of anomalies in the field of a body have been reported in the scientific and medical literature. In 1926, H. Fricke and S. Morse in an article “The electric capacity of tumours of the breast”, 1926 J. Cancer Res. 16 pp. 310-376 reported that the electrical properties of breast tumors differ significantly from healthy tissue. U.S. Pat. No. 4,539,640, issued Sep. 3, 1995, to inventors Bradley Fry and Alvin Wexler describes a method to solve electrical field equations that govern the flow of current in a conductive medium and extract an image of the interior of the medium. Other researchers have also been active in the effort to refine the technology, and improve the definition of tumors and distinguish between benign and malignant tumors. U.S. patent application Ser. No. 09/801706 filed Mar. 9, 2001 and U.S. Pat. No. 6,210,990 dated Mar. 13, 2001 are both directed to methods of refining the definition of tumors and the differentiation between benign and malignant tumors.

It is well known that malignant tumors differ in their electrical characteristics from normal tissues, and a variety of indices based on these characteristics may be utilized to distinguish a tumor from other tissues for diagnostic purposes. The distinguishing features may be differences in conductivity, dielectric constant, or similar characteristic; measured at one or more frequencies, and based on a value of the characteristic, or a ratio of values measured at different frequencies or similar comparison of values; or tumors may be distinguished by pattern recognition methods. For example, if it is known that a malignant tumor has four times the conductivity of normal tissue, an area of an image having such conductivity difference may be strongly indicative of that area being a tumor.

### SUMMARY OF THE INVENTION

The HDEIT method of the present invention permits one to use a variety of such indices to distinguish a tumour from normal surrounding tissue because it produces the value of the tissue characteristic at each zone in the tissues measured in accordance with the applied frequency. The tumour distinguishing analysis may be applied to the HDEIT image, or may be applied to the data that comprise the image without generating the image. Such methods are intended to permit the detection of tumors that are too small to be accurately seen in an image, but produce a large enough index for diagnostic purposes. One can apply this capability of the HDEIT method in a number of ways. For example, one can quickly scan the breast at low resolution, perform a distinguishing analysis for tumors, and then only perform a longer-duration high resolution scan if there was an indication of a diagnostically significant area to be examined. A number of similar applications will be apparent to those skilled in the art.

HDEIT is a method of imaging the internal tissues of the body, or a specific part of the body. HDEIT makes electrical measurements at the surface of the body, and solves the field equations to produce a three-dimensional image of the internal tissues by their differing electrical properties. The HDEIT method uses the algorithm described in Fry and Wexler U.S. Pat. No. 4,539,640 (referred to as “the Basic Algorithm” hereafter), and the acceleration and image-processing methods described herein, to permit much more detailed resolution than do other EIT methods. The method is safe, non-invasive, simple, and is a better diagnostic tool for distinguishing malignant tumors in the breast than other imaging methods.

The HDEIT method is also applicable to geophysical imaging, landmine imaging, mineral prospecting and other non-biological applications.

### BRIEF DESCRIPTION OF THE DRAWINGS

FIG. 1 is a block diagram of a system of the present invention,

FIG. 2 is a flow diagram of the MPC method;

FIG. 3 is a representation of a cube for approximating a breast,

FIG. 4 is a representation of the location of a tumor in FIG. 3,

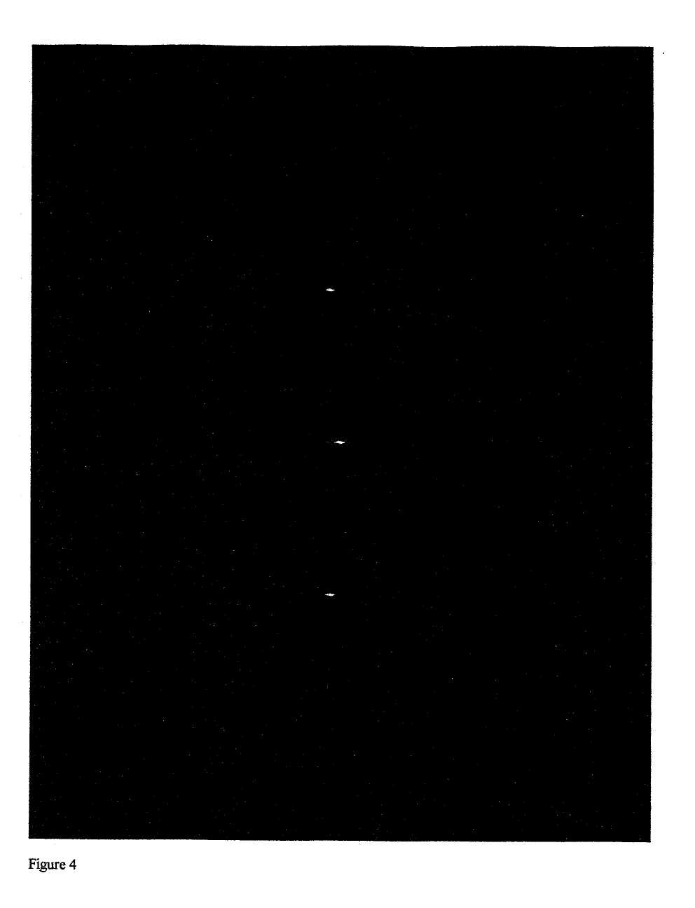

FIG. 5 is a flow chart of the LC method, and

FIG. 6 is a representation of a breast with a benign and a malignant tumor.

### DETAILED DESCRIPTION OF THE INVENTION

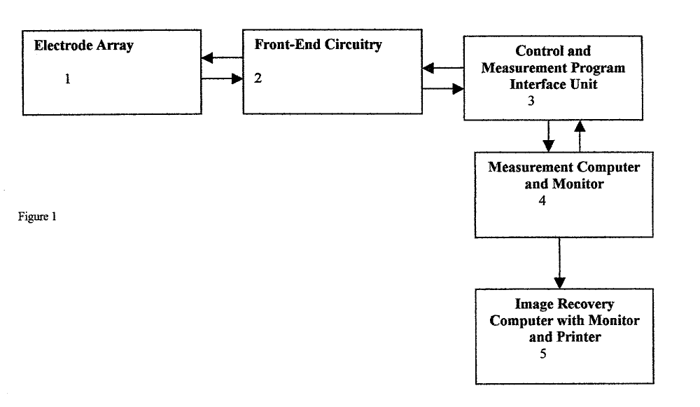

FIG. 1 is a block diagram of a system on which the present invention can be implemented. The system is comprised of a combination of circuits and electronic devices controlled by a computer as detailed below. The HDEIT imaging system consists of measuring system elements 1, 2, 3, and 4, and an image recovery computer 5. The system elements are shown in FIG. 1. The electrode array 1 surrounds the breast and provides means for making electrical connections to the breast. Example methods are: the electrodes may be applied directly to the skin or electrically connected to the skin via a conductive fluid. Other methods are discussed below. The front end circuitry 2 comprises means to inject and withdraw safe low values of current at pre-set frequencies (typically in the range of 10 kHz to 500 kHz) through a selected pair of electrodes at a time; and means to measure the resulting voltages at other selected electrodes for each current injection. The control and measurement program interface unit 3 controls the operation of the front-end circuitry. It transmits control signals to the front end, and receives measured values from the front-end circuitry. The number and sequence of selected current injection electrode pairs and voltage measuring electrodes, the values of current and the frequency are pre-programmed for the test in the measurement computer 4. The measured data is stored in memory in the computer 4. The measured data of surface voltage measurements and injected currents is transferred to the image recovery computer 5. The HDEIT image recovery algorithm described herein is implemented on the image recovery computer 5.

The electrode array 1 may be made in several forms. It may be a flexible printed circuit board array of physiological electrodes with conductive gel arranged in a cup-shaped arrangement for direct application to the skin of the breast. Or it may be a cubic or cylindrical insulating container of electrolyte material with the electrodes in fixed positions on the inside surfaces of the container, and the breast pendant in the container, as detailed below. It should be noted that the HDEIT method permits imaging of the breast without the electrodes being directly applied to the surface of the breast, while other EIT methods do not provide this capability. For breast imaging, electrodes should almost surround the object to be imaged, thus making it easier to achieve clear results The number and configuration of the electrodes is selected according to the test to be performed.

The front-end circuitry 2 measures voltages on selected electrodes, and sends or receives currents through selected electrodes. Currents may be injected or removed through any electrode pair, and voltage measurements may be at any selected electrodes. Currents at different frequencies may be simultaneously passed through the electrodes or only a single frequency at a time. The circuitry includes means for selecting electrodes and the function of the circuitry associated with each electrode. The number and arrangement of the circuitry channels will be selected appropriate to the test being conducted.

The Control and Measurement Program Interface Unit 3 of the HDEIT system controls the front-end circuitry. It comprises means to provide power for the circuitry, the frequency drive to the current sources, the control signals sent by the computer to the front-end circuitry, to measure and process the measured voltage values, and to transfer the measured data to the computer, as well as with any required measurement and signal conditioning functions.

In this manner, current is applied to plural places on the surface of the medium, and current is received from other plural places on the surface of the medium after passing through the medium. The interface unit converts the current to digital form, and is measured with the voltage. Breast imaging should have electrodes almost surrounding the object to be imaged, thus making it easier to achieve clear results.

The computer 5 processes the signal received from the electrodes 1 in accordance with processes based on U.S. Pat. No. 4,539,640 issued Sep. 3, 1985, invented by Bradley Fry and Alvin Wexler. This procedure is referred to as the Basic Algorithm in the following discussion.

The Basic Algorithm error-function minimization method—on its own—requires a large number of iterations to produce an image with sharp edges. What it does produce is an image with “peaks”, “hills ”, “valleys” and “wells” of conductivity corresponding to the location of objects. These objects appear regardless of whether the computation is performed for several iterations or for several hundred iterations. The conductivity improvement directions are defined at a very early stage of computation, picking the local maxima or minima and locating these objects accordingly. These objects are hereinafter referred to, generically, as “peaks”. The method in accordance with an embodiment of the invention modifies the derivation of images of the element conductivities with an acceleration scheme applied within the peak regions.

In an attempt to improve the original Basic Algorithm, Condamines and Marsili (A New Version of Wexler Algorithm in Electrical Impedance Tomography, T. Condamines and P. M. Marsili, Conference on Inverse Problems of Wave Propagation and Diffraction, INRIA, 1996) observed that the Basic Algorithm provides good qualitative results but is quantitatively less accurate. They showed that perturbing the conductivity, in a suspect region, could yield improved results.

In accordance with the invention described in U.S. Pat. No. 4,539,640 issued Sep. 3, 1995, to Fry et al, in U.S. Pat. No. 6,201,990 issued Mar. 13, 2001 to Wexler et al, and in U.S. patent application Ser. No. 09/801,706 filed Mar. 9, 2001—the Peak Detection Method—upper and lower thresholds are applied to the values resulting from the processing at various locations of the region within which imaging is being performed.

The speed of the error function minimization method may be accelerated by predicting some of the element conductivities according to differences obtained in the early stages of an image recovery procedure. This aforementioned invention determines where the prediction or acceleration should be applied, by use of peak detection. The method is initially “trained” by an approximate solution evolving soon after the method begins. Instead of checking conductivity changes for each element, this method takes the entire body as a whole and finds the areas where objects are most likely to exist. Simulation results show great improvements in the speed of convergence and quality of images in cases where adequate contrasts exist between the background and objects.

In accordance with the peak-detection method embodiment of that invention, a definition of the neighbourhoods of the “peaks” is obtained, to which the acceleration method is applied. The boundaries are ill-defined by a straightforward application of the Basic Algorithm error-function minimization method. In accordance with the Peak Detection Method embodiment, a threshold criterion is utilized, between low and high-value regions, to determine boundaries within which acceleration procedures are applied. This boundary location is successively improved as the image definition is refined. The result is that edges are sharpened and the regions to be detected and displayed are more clearly demarcated.

It should be noted that the peak-detection method is a digital image processing procedure that will sharpen images but could have the deleterious effect of causing a divergence from physical principles that are assured by a strict solution of the Laplace equation. In order to avoid this effect, we use the peak-detection method in conjunction with the Basic Algorithm thus ensuring that the electromagnetic field equations are properly satisfied and that the current-flow paths are accurately determined. This permits (given that efficient methods are employed) very high definition images to be rapidly achieved. Indeed, by the use of regular finite elements, this approach can be generalized to use other object-dependent, image-processing methods between EIT iterations. This facilitates an enormous increase in processing speed as well as very rapid image resolution.

Another method to accelerate the evolution of the conductivity image pattern, also based upon studying the history of convergence, is described in the aforesaid U.S. patent application Ser. No. 09/801,706. This Multi-step Extrapolation Method tracks the displacement value, at each node, pixel or voxel at which the conductivity is calculated. Then, a number of functions are examined to find the one (called the characteristic equation) that adequately describes the behavior of the displacement norm as a function of iteration count. The numerical values of the coefficients or parameters, within the characteristic equation, are determined by fitting the equation to the data. Then the conductivities are determined by extrapolation to a large or infinite value of the number of iterations. Then the Basic Algorithm is employed to reassert the physics. Once a set of new and more accurate conductivity values results from this procedure, the procedure may be repeated as many times as required.

Measurement sets (described as excitations) are obtained by using pairs of electrodes as current electrodes and a selection of the remaining ones are potential measurement electrodes. Because a unique interpretation is not possible from the results of a single excitation, a number of linearly independent excitations are employed. In theory, a gradient optimization scheme, or a Newton-Raphson scheme, could be used to adjust an assumed internal conductivity distribution in order to minimize the difference between the calculated and the measured voltages over the surface. One disadvantage to these schemes is that such procedures produce dense matrices of order corresponding to the number of nodes employed. For problems with more than a few dozen nodes, this optimization procedure becomes impossibly lengthy. Fine definition cannot be achieved in this way. Attempting to control the interior conductivity distribution from the outer surface (i.e. remotely) results in an ill-conditioned system with consequent numerical instabilities. This is akin to controlling the position of the long end of a meter stick with a fulcrum 1 cm from the short end.

A common element, in the methods described herein is that rather than controlling the image evolution from the outer boundary, the problem is cast into the interior. That is, the residual to be minimized is defined over the interior of the region within which the imaging is to be performed rather than over the outer boundary surface. This is accomplished by using the boundary conditions (as well as the enclosed conductivity distribution at each stage of the iterative process) to solve for and define the interior current flow and potential distribution patterns. Then, as described in the following, an interior residual function is defined that affords a local support that results in stability and sparse matrices.

The Basic Algorithm

In operation, firstly two field solutions, one for each of the following boundary condition setups, are performed for each excitation pattern:

(a) Inhomogeneous Neumann boundary conditions are applied at each current-excitation point and homogeneous boundary conditions at points along the boundary where no electrodes are applied and with a reference ground potential applied at one or more points; and

(b) Dirichlet boundary conditions, with measured voltage values and with a reference ground potential are applied at one or more points and with inhomogeneous boundary conditions applied at each current-excitation point.

For convenience, these field solutions are termed the Neumann and Dirichlet solutions respectively. The field solutions are found through the solution of the Poisson equation:

−∇·κ∇φ=f (1)

where k, φ and f are the conductivity, electrical potential and impressed current source distributions respectively within the region being studied. The units are (ohm−m)−', volts and Amperes/m³, respectively.

Although, strictly speaking, this equation holds only for the d.c. case, it is applicable to the a.c. case if the conductivity is sufficiently high so that the dielectric effects may be ignored. The Poisson equation is subject to the following Neumann and Dirichlet boundary conditions, which are respectively:

$$κ ( s ) \frac{\partial φ}{\partial n} {\vert}_{s} = h ( s ) \\ φ ( s ) = g ( s ) \qquad ( 2 )$$

where h(s), in Amperes/m², describes the electrical current flux density entering or leaving the medium over an electrode surface. Where no current is impressed, h(s)=0.

φ(s) corresponds to the measured potential (in volts, V) over the surface.

Then Equation (1) is applied to each such pair of solutions for each excitation pattern. However, with boundary conditions corresponding to actual measurements and with the conductivity only an estimate of what actually existed during the measurement, the pair of boundary conditions applied to the solution cannot he expected to produce identical computed internal fields. The imposition of Ohm's law

\overline{J}=31 κ∇φ (3)

where J represents the current density over the interior region employing both the previously estimated current flow density and potential for all excitations permits a conductivity distribution to be found that yields approximate compatibility of the Neumann and Dirichlet boundary conditions to be attained. To this end, a least-square technique is employed to produce an improved estimate of the conductivity distribution—one that satisfies both boundary conditions, for all excitations, in an average sense. Thus displacement of the conductivity estimate is caused.

With the current density (as calculated from the potential using the Neumann boundary condition throughout) and the potential (as calculated using measured voltages applied, i.e. the Dirichlet boundary condition where appropriate), Ohm's law is generally not satisfied. Thus, there is a residual wherever J+kÑf is evaluated. To enforce compatibility, the minimization of the square of the residual over all points and for all excitations is sought. It is therefore sought to minimize

R=Σₓ∫∫∫ᵥ(\overline{J}+κ∇φ)·(\overline{J}+κ∇φ)dv (4)

where R is the squared residual sum. V is the region over which the imaging is performed, and X represents the excitations over which the sum is taken.

Because Equation (4) can be represented as a summation over finite element volumes Vj, it can be written as

R=ΣₓΣⱼ∫∫∫ᵥⱼ(J+κⱼ∇φ)·(J+κⱼ∇φ)dv (5)

Then, to minimize the residual by adjustment of each conductivity value, we take

$$\frac{\partial R}{\partial {κ}_{i}} = 0 \qquad ( 6 )$$

in which J and f are held at previously computed values. Then, upon rearranging the equation,

$${κ}_{i} = \frac{- {\sum }_{X} \int \int {\int }_{{V}_{i}} \overline{J} \cdot ∇ φ ⅆ v}{{\sum }_{X} \int \int {\int }_{{V}_{i}} ∇ φ \cdot ∇ φ ⅆ v} \qquad ( 7 )$$

Several numerical methods may be used to accomplish the above operations consisting of the solution of two sets of voltage potential fields for each excitation (i.e. one with the Neumann boundary condition and the other with the Dirichlet boundary condition applied where appropriate) followed by the evaluation of an estimate of the conductivity distribution . We have used the finite element method (FEM). In its simplest form, one may assume a constant k value within each element i. However, the algorithm provides for inhomogeneous and orthotropic conductivity values. If the conductivity is allowed to vary within each element, the conductivity value would then need to be evaluated at several points within each element.

Equation (7) results in a very sparse matrix operation as a result of using the Finite Element Method over the interior. (This is in contrast to attempting to match the measured and computed boundary potentials by optimization of the interior voxel conductivities, a method that produces unwieldy and dense matrices and ill-conditioning.) Sparse matrices permit a great number of variables to be managed. In this case, this means that we are able to deal with small pixels thus yielding high definition imaging on the basis that the number of k updating iterations is not large.

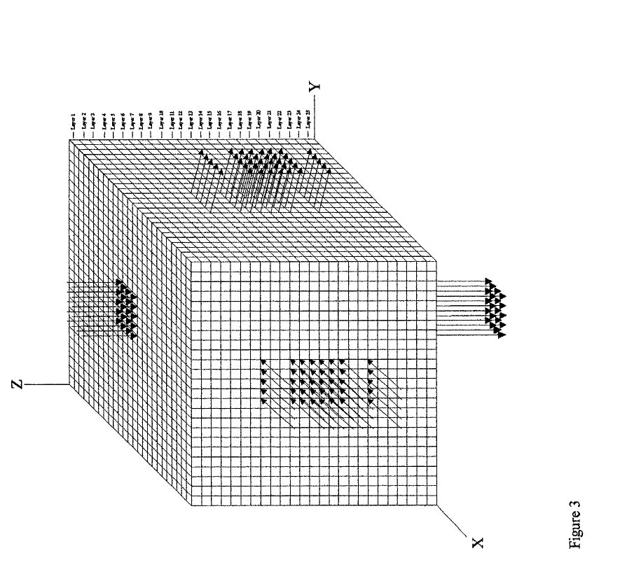

As an example, consider that a three-dimensional grid of nodes (FIG. 3) is defined over a cube considered to be excised from the host medium and including the region of interest. The cube is subdivided into a mesh defined by n points per edge. Thus there are n-1 links along each dimension and hence (n-1)³ k voxels (i.e. volume pixels) within the region.

To achieve 2 mm resolution, one must have voxels of approximately 2 mm to a side. A cube, of 10 cm to a side, would have 50³=125,000 voxels to attain this goal. The sparse field-solving and conductivity matrix systems would be of this order and they are individually computationally manageable.

Although the inverse problem is nonlinear, the requirements of linear algebra offer a guideline as to the number of measurements needed. Assume that the system has 400 electrodes that may be alternatively employed as active sources and passive receivers. This means that 399 linearly independent excitations are available. With two active electrodes, for each excitation, and never taking measurements at electrodes that are then active (and thus eliminating contact and spreading resistance errors), and allowing for one reference ground point, measurements may be made at 397 nodes for each excitation. This results in close to 160,000 linearly independent measurements which yields an overdetermined system.

We are at liberty to employ an electrode system that contains a number to electrodes that are alternatively active current sources and voltage measurement sites and a set that are used only as voltage measurement sites. As long as the resulting number of equations somewhat exceeds the number of unknown conductivity values, one is assured that imaging may be accomplished. In this manner, the number of excitations impressed upon the object may be significantly reduced if the number of voltage measurement sites is sufficiently large. Thus the number of field computations is similarly reduced and so is the time to uncover the image.

To increase the speed of the image processing a variety of methods, based on a study of the convergence history pattern, may be applied to accelerate convergence. These may be used individually or in collaboration with one another to attain desirable results.

The Modeler-Predictor-Corrector (MPC) Method

In accordance with another embodiment, the displacements of the electrical potential values (and, optionally, the conductivity values as well) at each conductivity-calculation stage is tracked. The displacement values, at each node at which the conductivity is calculated, are evaluated and stored. Then, a function is found that satisfies the least-squared difference (i.e. the residual) in an optimal sense. In other words, the least-squared residual (often called the error) is minimized. The improvement over the Multistep Extrapolation Method is that estimation of potentials and conductivities, with this approach, is likely to be closer to physical reality by virtue of the averaging effect (over the electric potentials and conductivities) rather than to base an extrapolation, with iteration count, upon a single variable (i.e. the conductivity) alone. In this way, it is found that the number of iterations required is usually significantly less than that needed for the Multistep Extrapolation Method. An example method follows:

Step 1: Model Φ and k as a Function of Iteration i.

The recovered potential and conductivity distributions, over the first 2^{nd} to n iterations, are modelled using the nonlinear least-squares fitting scheme of Levenberg-Marquardt (LM) (W. H. Press, B. H. Flannery, S. A. Teukolsky and W. T. Vetterling, Numerical Recipes in C, Cambridge University Press, Cambridge, 1988, pp. 542-547). See also, High Definition Electrical Impedance Tomography, CIP application Ser. No. 09/801,706.

The fitted mathematical equations as a function of iteration i, are expressed as:

φ=φ(i) (8)

κ=κ(i) (9)

where Φ and K are the potential (i.e. voltage) and conductivity distributions at iteration i respectively.

In order to use the Levenberg-Marquardt least-squares fitting algorithm and to ensure correct “fit parameters”, approximations to the actual physical shapes of the potential and conductivity distribution histories, over all voxels (i.e., the fit functions), are required. By studying the convergence patterns, it appears that the equations

y=a−bln(x+c) (10)

And

$$y = {ae}^{\frac{b}{x + c}} \qquad ( 11 )$$

(Along with Many other Possibilities) form Appropriate “fit functions”. The variable x would represent the iteration count and y the conductivity k, potential f, or directional derivatives of the gradient Ñf, i.e. the derivatives in the x, y and z directions. The Levenburg-Marquardt algorithm determines the a, b and c parameters.

Step 2: Predict k at Iteration I

Once the optimal equations for potential, φ, and conductivity, k are derived from Step 1, the potential relation (8) is then used to derive the iteration number I, at which the algorithm is considered to have converged. This is accomplished by substituting the known potential distribution, φ_{known}, obtained through direct measurements, into the mathematical relation (8). Rather, the known potential φ_{known} is used to derive the correct iteration number, I for which the algorithm is considered to converge.

φ_{known}=φ(I) (12)

κ_{d}=κ(I) (13)

One preferred method is to seek the iteration number for which the root-mean-square or sum of absolute values of the deviation from the known boundary potentials is minimised.

The method permits the extrapolation of the conductivity k, potential φ, or directional derivatives of the gradient∇φ, i.e. the derivatives in the x, y and z directions.

Thereafter, the derived number of iterations, I, is used in the conductivity relation (13) to derive the corresponding conductivity distribution k_{d}. At this stage, it is assumed that the derived conductivity distribution at iteration I is equivalent to or close to the distribution being sought. The algorithm then goes on to Step 3

Step 3: Correct Conductivity by Re-Initializing

The earlier assumption of Step 2, that the derived conductivity distribution k_{d} at iteration I is equal to the distribution being sought is not entirely incorrect. It is obvious that k_{d} was arrived at by some interpolation operations, quite likely it contains some error. To minimize the errors that might have been introduced at Steps 1 and 2, the derived conductivity distribution k_{d} is corrected by re-initializing. Re-initialization involves using the derived conductivity distribution k_{d} as the initial starting conductivity distribution in Step 1 of the original basic EIT algorithm flow chart. As well as removing any discrepancy, the re-initialization step assures the correct physical approach of the original basic EIT image reconstruction algorithm.

For the preceding, Equation 7, we may choose to employ extrapolated values of J (i.e. using the Neumann boundary conditione-derived potentials) and (f using the Dirichlet boundary condition-derived potentials).

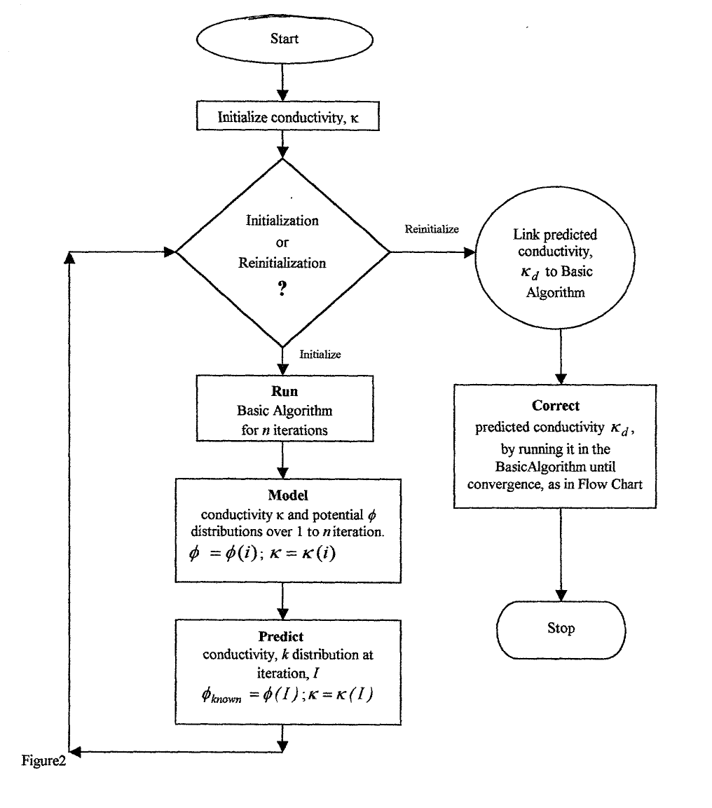

FIG. 2: is an MPC flow chart. The algorithm is initialized using an assumed conductivity distribution. A decision is then made if it is an initialization or re-initialization procedure. If it is initialization, the algorithm proceeds to the Run, Model, and Predict steps respectively. The derived conductivity k_{d}, at the end of the Predict stage, gets fed back to the decision phase. At this point, the algorithm recognizes that this is the re-initialization step. This in turn is fed to the basic algorithm for error correction until convergence. After the initialization or re-initialization, the algorithm proceeds to the Run, Model, and Predict steps respectively. The derived conductivity k_{d}, at the end of the Predict stage, gets fed back to the decision phase. At this point, the algorithm recognizes that this is the re-initialization step. This in turn is fed to the Basic Algorithm for error correction until convergence.

The Locator-Compensator (LC) Algorithm

The Basic Algorithm locates the region(s) of interest (e.g. a tumour at a very low iteration count, irrespective of the size and type, i.e. whether it is benign or malignant). However, the recovered conductivity value, at such early iteration, is frequently inaccurately determined.

For EIT to be of clinical value and for an accurate diagnosis for, say, breast cancer, it ought to be able to detect abnormalities at an early stage, physically equivalent to an approximate size of 2-4 mm. Thus, it appears that the spatial resolution that can be obtained with the original basic algorithm is not suitable to image small breast tumours. The Locator-compensator (LC) algorithm was developed and implemented for computer simulations of small tumours. This improved the Basic Algorithm's spatial resolution and its ability to detect small breast lesions and to define edges with precision at speed. For example, measurements are taken on five sides of a cube (FIG. 3) representing an approximate model of the breast. The remaining side represents the side of the breast that is attached to the chest wall.

Using an iterative method, to solve the nonlinear k-imaging problem, requires a number of iterations that increases with the number of mesh links. Intuitively, this may be understood as error has to migrate to the outer boundary and the number of k iterations needed increases with the number of mesh links to the boundary. This is consistent with the observation that deeply-imbedded objects suffer from less clarity, at any given iteration count, than those objects nearer the surface. In effect, these regions have a tendency to be modeled by equivalent circuitry with slow improvement of the actual image recovery only with a greatly increased number of iterations.

To obtain a high degree of image quality (including high-resolution boundary definition) and to determine the conductivity accurately with a very fine mesh, it is useful to determine the region of an object and to surround it with a reasonably good estimate of the local boundary conditions. Then, the migration of error to the local boundary happens more rapidly with iteration count. A theoretically strict approach would involve mesh grading so that finer meshes may be built up in the vicinity of objects of interest. But this would increase the required iteration count demanded. As it happens, the global potential distribution settles rather rapidly and—although there may be some error—the averaging effect over the local boundary and the error minimizing effect of the least-square Basic Algorithm compensate somewhat in the course of image recovery. With this in mind, a number of options present themselves.

With a reasonable approximation to the local boundary conditions, iterations may proceed within the local region alone. Here, we consider a compromise that involves reasonably well-computed potentials (i.e. the Dirichlet boundary condition) surrounding the local region and the Neumann boundary condition maintained at the outer boundary and the iterations proceeding over the entire region of interest.

A variant of the peak detection method is used to locate the spatial coordinates of the peak(s) at early iterations. Once the coordinates of the peak(s) are localized, then the improved local image recovery (i.e., in terms of improved conductivity values and edge definition), are compensated. The compensation is performed by applying the Basic Algorithm, for the conductivity-updating scheme, over the localized regions rather than over the whole imaging region.

In this implementation of the peak detection method, rather than having to assume a percent of the background conductivity (i.e., *K_{b}), which subsequently is used as the basis for the criterion for peak detection (i.e., by comparing *K_{b} to ΔK), the variant of the peak detection method employs a non-subjective approach for peak(s) localization. The basic algorithm with its original conductivity updating-scheme is used to sweep though an imaging region for n iterations. The recovered conductivity distribution at each iteration (i.e., K₁, K₂, K₃ . . . , Kₙ) is averaged over n iterations (i.e., K₁+K₂+K₃+ . . . +Kₙ/n) The averaged conductivity distribution at n iteration (i.e., \overline{k}ₙ) is then averaged over all the elements within the imaging region. This results in an average conductivity per element (\overline{K}ₑₙ=\overline{K}ₙ/N), N being the total number of elements in the adopted mesh for image recovery (or solution of the inverse problem). Peaks are located by comparing \overline{K}ₑₙ to Kₑₙ₊₁, to where kₑₙ₊₁, is the conductivity of each element at n+1 iteration. If Kₑₙ₊₁>\overline{K}en, the element or an aggregate of element is/are identified as peak(s) or if Kₑₙ₊₁>\overline{K}en then the element or an aggregate of element is/are not regarded as peak(s). To avoid detecting any unwanted anomalies due to truncation or discretization, a filtering scheme is applied prior to peak(s) detection. Once elements) is/are identified as peak(s) (or troughs), the spatial coordinates of the immediate elements surrounding the peak(s) element(s) are identified. It is in the vicinity of these coordinates that the new conductivity distribution updating-scheme is implemented to compensate for loss in spatial resolution.

Once identified, the calculated potentials at coordinates of identified element(s) are then substituted by the calculated interior potentials obtained from measured or known surface potentials. These calculated potentials applied to nodes surrounding the peak(s), applied as boundary conditions, cause the local-region interior potentials to converge. Since the localized region(s) would generally consist(s) of a few elements (i.e., pixels or voxels), this selective conductivity-updating scheme is relatively fast and effective. While conductivity in the localized region is updated with the localized updating scheme just described, conductivity updating, for the rest of the imaging region, proceeds via the original basic algorithm conductivity-updating scheme. This process continues until measured and calculated potentials are equal (i.e., until convergence is attained).

The Locator-Compensator method is summarized in the following steps:

Step 1: Run the Basic Algorithm for n Iterations.

The original Basic Algorithm, with its conductivity updating scheme, is used to sweep through the imaging region for n iterations. The revised estimate of conductivity within element i over all node points and over all excitations is

$${κ}_{i} = \frac{- {\sum }_{X} \int \int {\int }_{{V}_{i}} \overline{J} \cdot ∇ φ ⅆ v}{{\sum }_{X} \int \int {\int }_{{V}_{i}} ∇ φ \cdot ∇ φ ⅆ v} \qquad ( 14 )$$

Where \overline{J} is the estimated electrical current density distribution, Φ is the potential obtained with Dirichlet boundary conditions, k₁ is a revised estimate of the conductivity within element i, v₁ is the volume of the element i, and X represents the excitations over which the sum is taken.

Step 2: Save the Conductivity Distribution k Over 1 to n Iterations.

The recovered conductivity distribution over the period of iteration 1 to n is saved and averaged over n iterations, \overline{K}ₙ. The average conductivity distribution \overline{K}ₙ is then averaged over all the elements, \overline{K}ₑₙ=\overline{K}_{n/M}. The average over all elements \overline{K}ₑₙ is then used as a criterion component for peak(s) detection.

Step 3. Locate peak(s) with the Peak Detection Variant Method at n+1 Iterations.

Peak(s) is/are located by comparing \overline{K}ₑₙ to Kₑₙ₊₁, where Kₑₙ₊₁, is the conductivity of each element at n+1 iteration. If Kₑₙ₊₁>\overline{K}ₑₙ, the element or an aggregate of element is/are identified as peak(s) or if Kₑₙ₊₁≦\overline{K}ₑₙ, then the element or an aggregate of element is/are not regarded as peak(s). To avoid detecting any unwanted anomalies due to truncation or discretization, a filtering scheme is applied prior to peak(s) detection. Once the peak(s) is/are located, the immediately surrounding elements' nodes spatial coordinates are identified. The potentials at these nodes are saved accordingly.

Step 4. Compensate for Resolution at (n+2) to N Iterations.

When viewed as a whole, the imaging region can be considered to consist of localized peak(s) region and a background region. The conductivity updating-scheme for the background region is that as utilized by the Basic Algorithm. The new conductivity updating scheme for the localized region(s) can be arrived at by applying the Basic Algorithm conductivity updating scheme to the identified region(s) in much the same way. In this new approach, the calculated potentials at the spatial coordinates of the external nodes of the elements identified in Step 3 are substituted by the interpolated calculated potentials obtain from the measured or known surface potentials. That is to say applying Dirichlet boundary conditions to the localized regions while leaving the remaining initial (i.e., at n +1 iterations) Neumann boundary conditions unchanged. This causes the interior potentials to be nudged in the correct direction. This is performed for each iteration and over the whole imaging region iteratively until convergence at iteration N. Similarly, the Dirichlet boundary condition for the localized region(s) is,

φ(Is)=g(Is) (15)

Which corresponds to the interpolated calculated potentials at external nodes coordinates of element(s) identified in Step 3. In addition the boundary conditions must include the Neumann conditions at current-injection sites as previously described.

By applying the Basic Algorithm to the local region, the revised estimate of conductivity within element 1 over the volumetric region enclosed by the identified node points coordinates and over all excitations is given as

$${κ}_{l} = \frac{- {\sum }_{X} \int \int {\int }_{{V}_{i}} \overline{J} \cdot ∇ {φ}_{l} ⅆ v}{{\sum }_{X} \int \int {\int }_{{V}_{i}} ∇ {φ}_{l} \cdot ∇ {φ}_{l} ⅆ v} \qquad ( 16 )$$

Where \overline{J} is the estimated electrical current density distribution, Φ₁is the calculated potential obtained from the measured or known surface potentials (i.e., from application of Dirichlet boundary conditions), k₁ is a revised estimate of the conductivity within element 1 of the localized region, v₁ is the volume of the element 1, and X represents the excitations over which the sum is taken.

By combining Equations (15) and (16), a new conductivity-updating scheme is obtained (17). This revised scheme is applied to each element in turn to update the conductivity distribution over the entire region within which the imaging is being performed.

$${κ}_{i + l} = \frac{- {\sum }_{X} \int \int {\int }_{{V}_{i}} \overline{J} \cdot ∇ φ ⅆ v}{{\sum }_{X} \int \int {\int }_{{V}_{i}} ∇ φ \cdot ∇ φ ⅆ v} + \frac{- {\sum }_{X} \int \int {\int }_{{V}_{i}} \overline{J} \cdot ∇ {φ}_{l} ⅆ v}{{\sum }_{X} \int \int {\int }_{{V}_{i}} ∇ {φ}_{l} \cdot ∇ {φ}_{l} ⅆ v} \qquad ( 17 )$$

The Locator-Compensator algorithm makes use of the combined updating scheme (17) to recover the conductivity distribution. Characteristically, the fact that the original basic algorithm locates the peak(s) at early iteration, application of the LC algorithm, will in theory, ensure that the peak(s) converge(s) much faster and with adequate resolution at diseased-to-normal tissue interface.

Should the applied boundary condition, that surrounds at the local region, not be sufficiently accurate, the procedure may be repeated using the newly-computed conductivities. This is akin to a block-iterative scheme.

The LC method involves locating the peaks with a variant of the peak detection image processing algorithm and subsequently applying the new conductivity-updating scheme. This improves the resolution at diseased-to-normal tissue interface.

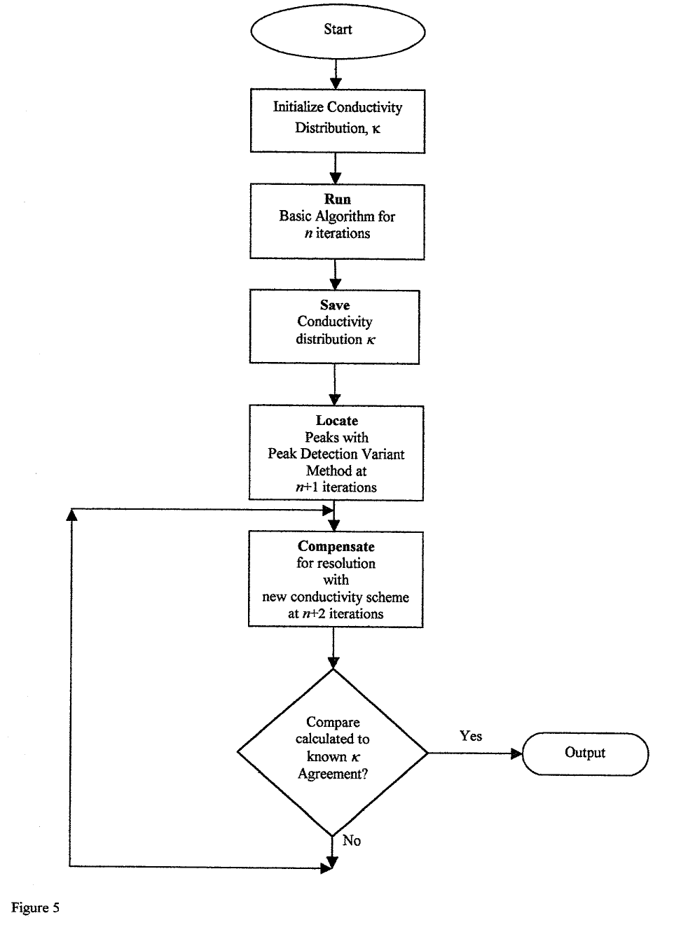

FIG. 5:is the Locator-Compensator (LC) flow chart. The algorithm is initialized using an assumed conductivity distribution. The original basic 3-D EIT algorithm is allowed to run for n iterations. The algorithm then proceeds to the Save, Locate, and Compensate steps. A comparison is then made to the known potential distribution and, if results agree, then the algorithm outputs. If not, the algorithm keeps iterating through the compensation step until convergence is complete. If the convergence criterion is not satisfied, the total region is then iterated and the local region reconstructions are repeated.

The Combined MPC and LC Algorithm

The combination of the MPC and LC algorithms, together with the Basic Algorithm, is beneficial. Generally, the MPC helps to improve the convergence rate overall and to rapidly identify peaks warranting further inspection. And the LC improves resolution, particularly at diseased-to-normal tissue interfaces, and results in accurate determination of conductivity values within suspect regions thus assisting diagnosis.

A preferred embodiment of the HDEIT method is to use the MPC algorithm employing several iterations of the re-initialization loop to bring the image to an acceptable level of convergence without pressing it to its limit. This then uncovers peaks (especially highly conductive regions) that bear further examination and with enhanced LC processing yields refined images and accurate conductivity values within defined local regions.

The LC method is applied by defining local regions. These regions may be identified by inspection or automatically by considering contiguous regions within which the LC method can be applied sequentially. Moreover, the process of scanning the entire global imaging region may be repeated several times, in a block-iterative manner, to achieve highly-accurate image definition.

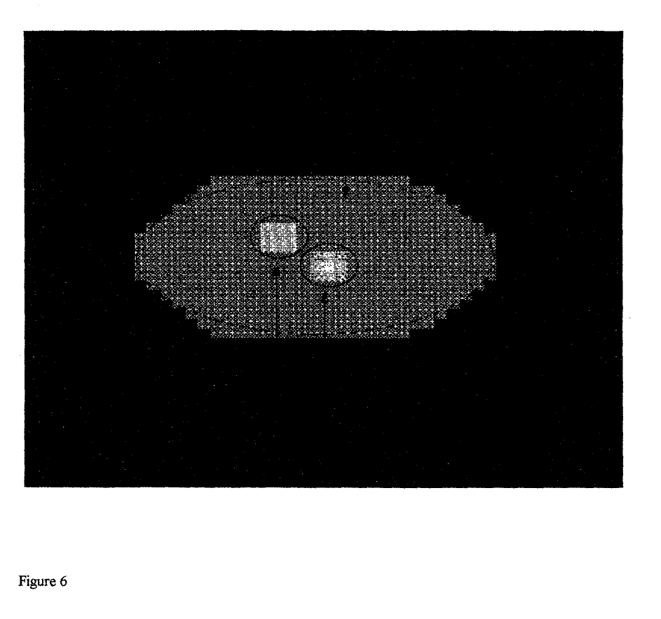

In summary, this improved algorithm, consisting of the MPC and LC methods in conjunction with the Basic Algorithm, proceeds in the manner described in the following and as shown in the flow chart FIG. 6.

Step 1: Run the Basic Algorithm for n Iterations.

The EIT Basic Algorithm, with its conductivity updating scheme, is used to sweep through the imaging region for n iterations in order to determine the behavior of the convergence pattern in the course of solving the imaging problem as governed by physical principles.

Step 2: Run the MPC Algorithm for m Iterations.

From the history of the recovered potential and conductivity (and, additionally if desired, the components of the gradient) distributions of Step 1, the MPC algorithm is applied to model, predict, and correct for a derived conductivity distribution, k_{d}. The correction is performed using the Basic Algorithm, to reassert the physics, and this is performed for m iterations.

Step 3: Run the LC Algorithm for p Iterations.

Following Step 2, the Locator-Compensator (LC) algorithm is applied. This locates the peaks and the revised conductivity distribution scheme is applied subsequently to update conductivities appropriately (i.e., for localized and background regions). The LC algorithm is applied for over p iterations. This process continues until the calculated and measured potentials are acceptably within agreement. It is then deemed that the process has converged.The algorithm then outputs the recovered conductivity image. The whole process takes approximately c (c=n+m+p) iterations to converge.

Distinguishing Tumor Signatures

It is well known that malignant tumors differ in their electrical characteristics from normal tissues, and a variety of indices based on these characteristics may be utilized to distinguish a tumor from other tissues for diagnostic purposes.

The distinguishing features may be differences in conductivity, dielectric constant, or similar characteristic, measured at one or more frequencies, and based on a value of the characteristic, or a ratio of values measured at different frequencies or similar comparison of values, or tumors may be distinguished by pattern recognition methods.

A tumor that is too small to image on its own will cause an apparent (but diminished) conductivity increase in adjacent voxels. For example, if it is known that a tumor has four times the conductivity of normal tissue, an area of an image having an increased conductivity may be strongly indicative of that area containing a tumor. The HDEIT method permits one to use a variety of such indices to distinguish a tumor from normal surrounding tissue because it produces the value of the tissue characteristic at each zone in the tissues measured in accordance with the applied frequency. The tumor-distinguishing analysis may be applied to the HDEIT image, or may be applied to the data that comprise the image without generating the image. Such methods may permit the detection of tumors that are too small to be accurately seen in the image, but produce a large enough index for diagnostic purposes. One could apply this capability of the HDEIT method in a number of ways.

For example, one could quickly scan the breast with a lower-resolution and simplified machine, perform a distinguishing analysis for tumors, and if there is an indication of a diagnostically significant area to be examined, only then perform a longer-duration high resolution scan, with an enhanced equipment. Or, the image could be automatically marked to direct the physician's attention to the regions likely containing a tumour. A number of similar applications are obvious.

The work carried out and described here demonstrates clearly that the procedure is competent to image small breast tumors and to diagnose them to indicate whether they are malignant or benign.

Implementation of the combined MPC and LC algorithms, in conjunction with the Basic Algorithm, was demonstrated to improve both the convergence rate and the overall spatial resolution.

Conductive Fluid Connection to the Breast

The skin is a natural barrier protecting the inner tissues and presents a high impedance path to electrical contact with the tissues unless actions are taken to reduce this high skin impedance. There are several means of making the electrical connections between the electrodes and the surface of the breast.

Other EIT methods require the electrodes to be directly connected to the skin of the breast. One means is to fabricate the electrodes as an array on the inner surface of a breast cup-shaped electrode holder that is placed in intimate contact with the breast, or to use some other form of electrode holder to position the electrodes against the breast. Some applications have used an array of metallic pins protruding from a surface and held firmly against the breast, but this requires a skilled user and can be uncomfortable if too high a pressure is applied, others have used individually-positioned electrodes. Such electrodes usually require pre-treatment of the skin to reduce the skin impedance or the use of a localized conductive medium between the electrode and the skin of the breast, such medium being constrained to prevent electrode-to-electrode electrical paths. These contact means require a means of keeping the electrodes in intimate contact with the skin. Contact may be achieved by fitting the electrode holder to the breast so that it supports the breast, or by use of a slight vacuum to hold the electrodes against the skin, or by pressing the electrodes to the skin. HDEIT method of the present invention may also use such means of electrode application.

The preferred implementation of the present invention permits the use of electrodes physically removed from the breast surface and making electrical contact to the skin through a pool of conductive fluid of appropriate conductivity arranged between the skin and the electrodes. One means of achieving this conductive fluid connection is to arrange the electrodes as an array on the inner surface of an insulating open-topped container fabricated in the shape of a round or oblong or rectangular cross-section with a flat or concave bottom, and a conductive fluid medium held in the container. The patient will lie prone on a suitable support means with the breast to be imaged pendant in the container so that the electrical connection between the skin of the breast and the electrodes is through the pool of conductive fluid medium. Thus there is no need to apply the electrodes directly to the breast. This is the preferred means of electrode configuration for HDEIT, since it offers several advantages permitted by HDEIT that other EIT methods may not permit.

Some of the advantages of this means of making the electrical connections through a pool of electrolytic medium are as follows. The position is comfortable for the patient. The pendant position allows the breast to be naturally stretched out for better visualization-particularly important for large breasts. The fixed electrode positions at the walls of the container enhance accuracy of the imaging process. The electrolyte medium is at a comfortable temperature, and the material composition of the electrolyte may be selected to optimize the electrical characteristics for electrical contact and HDEIT image acquisition. The size of the electrode array need only approximate the size of the breast, so the electrode arrays may be made in a limited range of sizes. The apparatus may be arranged so that both breasts may be imaged without repositioning of the patient. And, since patient positioning is so easy, user training and qualification is minimized.

The HDEIT process permits use of such a container and electrolyte combination as the algorithms involve the numerical solution of the field equations throughout the composite medium including the breast. In effect the entire system is treated as an extended breast region with the algorithm determining the conductivity distribution throughout. In so doing, because of conductivity contrast between the conductive fluid and the breast, HDEIT defines the shape of the breast without the necessity of external measurements being done.

In the investigation of known EIT methods it is common practice to make water-bath models of the human torso. It is common to physically model the body as a cylindrical shape by mounting an array of metallic electrodes into the inner surface of a vertical water-filled plastic cylinder with objects of known and different conductivity arranged inside the cylinder to represent the internal body organs. The water and the objects are generally made conductive by the addition of electrolytes. In this way the electrodes contact the surface of the “body:” modeled by the water bath. These methods are commonly used because the location and characteristics of the electrodes must be taken into account by the analysis methods that are being used.

The present invention allows for electrically connecting to the body from a set of electrodes physically separated from the body, and involves the body part immersed into a container of conductive fluid, with a regular array of electrodes immersed in the fluid in the container at some small distance from the body and surrounding the body part. Thus, there is no need to fit the electrodes to the surface of the body part. The body part is simply immersed into the bath of fluid with the electrode array surrounding it. The method is simple and convenient since the fluid bath will accommodate a range of sizes and shapes of body parts. The HDEIT method images the body part in the fluid as part of the fluid object so that the electrodes do not need to be applied directly to the skin surface of the body part. The entire region is imaged by virtue of it all being treated as an extended breast image. This adds extra distance between electrodes and objects (e.g. lesions) that are to be imaged. The HDEIT imaging method allows for this type of configuration because of the generality of the Basic Algorithm and the capability of the system to image objects remote from the electrodes. This indirect contact between the electrodes and the body through a bath of conductive fluid is unique for HDEIT

The container for the conductive fluid may be open at the top so that the body part is dipped down into the fluid in the container for the duration of the scan. For example, for imaging of the female breast, the container may be incorporated into an examining table so that the woman would lie face down on the table with the breast pendent into the container of fluid. Alternately, the container may be arranged as a vessel made up of several parts with leak-proof seals between them, that may be placed around the body part to be imaged, sealed to the body surface with a flexible seal means at one or both ends, and then filled with the conductive fluid. The electrode array may be arranged into the inner surface of the container holding the conducting fluid or be otherwise arranged in the container. The materials and parameters of the electrodes will be arranged to be suitable for imaging. The conductivity of the fluid will be arranged to be a value suitable for the purpose of imaging.

This invention differs from the usual EIT water bath modeling of the body since the body part immersed in the conductive fluid is separated from the electrode array in the fluid. In the laboratory modeling of the body by a water bath, the fluid of the bath is directly a part of the body model, with the electrodes making direct contact to it. In this invention, the conductive fluid surrounding the body part is just the electrical coupling medium between the body part and the array of electrodes.

A person understanding this invention may now conceive of alternative structures and embodiments or variations of the above. All those which fall within the scope of the claims appended hereto are considered to be part of the present invention.

We claim:

1. A method of imaging objects in a medium, the objects having specific impedances which are different from the specific impedance of the medium comprising,
a) applying an electrical current to the medium at various locations,
b) detecting voltages produced by the current which has passed through the medium from the surface of the medium at various locations,
c) iteratively repeating steps a) and b), changing the value and locations for applying the electrical current and measuring the voltages,
d) successively determining the region of the medium in which the objects are located with increasing accuracy by processing values of the detected voltages, using an algorithm to solve the field conditions in the medium, thereby determining a region in the medium in which the objects are located,
e) successively determining the location, shape, and conductivity of the objects with increasing accuracy by
i) determining a pattern of convergence,
ii) selecting a function which approximates the determined pattern,
iii) extrapolating the function for a predetermined number of iterations,
iv) determining the boundary conditions of the region of objects
v) repeating the iterations in the region defined by the boundaries, until convergence of the pattern and the values of voltage occurs, and
f) displaying the results graphically.

2. A method of imaging objects in a medium, the objects having a specific impedance which is different than the specific impedance of the medium comprising
a) initializing the conductivity distribution K,
b) running a basic algorithm for n iterations,
c) saving the conductivity distribution,
d) locating peaks with a peak detection method at n +1 iterations,
e) compensating for resolution with a new conductivity scheme at n+2 iterations,
f) comparing calculated with known K for agreement,
i) if agreement is obtained, output results,
ii) if no agreement is obtained rerun step e).

3. A method as claimed in claim 2, wherein between steps a) and b) the following process is carried out,
a1) determine if step a) is an initialization or a reinitialization,
a2) if a reinitialization linking predicted conductivity k_{d} to basic algorithm,
a3) correct predicted conductivity by running it in the basic algorithm unit until convergence, and
a4) output results,
a5) if step a) is not a reinitialization step,
a6) initialize by running the basic algorithm for n iterations,
a7) model conductivity k and potential φ distributions over 1 to n iterations,
a8) predict conductivity k distribution at iteration I, and then run steps b) to f).

4. A method as claimed in claim 3, wherein the basic algorithm comprises:
(a) applying electrical input currents at a plurality of selected current input sites of said structures, each of said electrical input currents flowing within at least one of said regions and exiting from said structure at a selected current output site thereof;
(b) measuring the voltages generated by each of said applied currents at a plurality of selected voltage measuring sites of said structure with respect to a voltage reference point, each of said selected voltage measuring sites being remote from the current input and output sites through which flows the current generating said voltages;
(c) calculating the voltages Φ at a plurality of locations within said structure, including said selected nit voltage measuring sites, with respect to said voltage reference point from the equation
−Δ₋ₖΔΦ=f,
Where k is a value of conductivity assumed for each of said locations and f is the density of each of the electrical input currents at said current input and output sites, the current traversing the surface of said structure except at said current input and output sites being assumed equal to zero;
(d) calculating the electrical current flux density \overline{J} at each of the locations for which the voltage was calculated in step (c) from the equation
\overline{J}=−k∇Φ;
(e) comparing the voltages calculated in step (c) for each of said selected voltage measuring sites of said structure and the corresponding voltages measured at said selected sites in step (b);
(f) repeating steps (c) and (d) when the difference between the voltages compared in step (3) are greater than a predetermined amount, the voltages measured in step (b) then being substituted at said selected voltage measuring sites for the voltages calculated in step (c);
(g) calculating new values for k for each of said locations when the squared residual sum R equals
$$\frac{\sum }{X} \int \int v ( \overline{J} j + k ∇ φ ) \cdot ( \overline{J} + k ∇ φ ) ⅆ v$$
Where V is the region over which the imaging is being performed and X represents the excitations over which the sum is taken, by determining the values of K which minimize R throughout said structure; and
(h) repeating steps (f) and (g) until the voltages compared in step (e) do not exceed said predetermined amount.

5. A method of detecting malignant and benign tumors in a breast comprising positioning an electrode array consisting of pairs of electrodes on the surface of the breast, passing current between selected pairs of electrodes sequentially, measuring the voltages between electrode pairs not carrying said currents,and calculating the position, size and malignancy of tumors from the potential and conductivity information derived from said voltage measurements.

6. A method of detecting malignant and benign tumors in a breast comprising positioning an electrode array consisting of pairs of electrodes on the surface of a dielectric container containing a conductive fluid, immersing said breast in said fluid, passing current between selected pairs of electrodes sequentially, measuring the voltages between electrode pairs not carrying said currents, and calculating the position, size, and malignancy of tumors from the potential and conductivity information derived from said voltage measurements.

7. A method of imaging an inhomogeneous or homogeneous medium and objects located therein, the objects having specific electrical properties which are different from the specific electrical properties of the adjacent medium comprising,
a) applying an electrical current to the medium at various locations,
b) detecting voltages produced by the current which has passed through the medium from the surface of the medium at various locations,
c) repeating steps a) and b), changing the value and locations for applying the electrical current and measuring the voltages,
d) successively determining the region of the medium in which the objects are located with increasing accuracy by processing values of the detected voltages, using an algorithm to solve the field equations in the medium, thereby determining a region in the medium in which the objects are located,
e) successively determining the location, shape, and conductivity of the objects with increasing accuracy by
i) determining a pattern of convergence,
ii) selecting a function which approximates the determined pattern,
iii) extrapolating the function for a predetermined number of iterations,
iv) determining the boundary conditions of the region of objects
v) repeating the iterations in the region defined by the boundaries, until convergence of the pattern and the values of voltage occurs, and
f) displaying the results graphically.

8. A method of imaging an inhomogeneous or homogeneous medium and objects located therein, the objects having specific electrical properties which are different than the specific electrical properties of the adjacent medium comprising
a) initializing the electrical properties distribution,
b) running the basic algorithm for n iterations,
c) saving the conductivity distribution,
d) locating peaks with a peak detection method at n +1 iterations,
e) compensating for resolution by applying the basis algorithm withiin the restricted region with a new conductivity scheme at n+2 iterations,
f) comparing calculated potential with measured potentials for agreement,
i) if agreement is obtained, output results,
ii) if no agreement is obtained rerun step e).

9. A method as claimed in claim 8, wherein between steps a) and b) the following process is carried out,
a1) determine if step a) is an initialization or a reinitialization,
a2) if a reinitialization linking predicted conductivity k_{d} to basic algorithm,
a3) correct predicted conductivity by running it in the basic algorithm unit until convergence, and
a4) output results,
a5) if step a) is not a reinitialization step,
a6) initialize by running the basic algorithm for n iterations,
a7) model conductivity k and potential Φ distributions over 1 to n iterations,
a8) predict conductivity k distribution at iteration I, and then run steps b) to f).

10. A method as claimed in claim 9, wherein the basic algorithm comprises:
(a) applying electrical input currents at a plurality of selected current input sites of said structures, each of said electrical input currents flowing within at least one of said regions and exiting from said structure at a selected current output site thereof;
(b) measuring the voltages generated by each of said applied currents at a plurality of selected voltage measuring sites of said structure with respect to a voltage reference point, each of said selected voltage measuring sites being remote from the current input and output sites through which flows the current generating said voltages;
(c) calculating the voltages (at a plurality of locations within said structure, including said selected voltage measuring sites, with respect to said voltage reference point from the equation
−Δ₋ₖΔΦ=f,
Where f is a value of conductivity assumed for each of said locations and f is the density of each of the electrical input currents at said current input and output sites, the current traversing the surface of said structure except at said current input and output sites being assumed equal to zero;
(d) calculating the electrical current flux density \overline{J} at each of the locations for which the voltage was calculated in step (c) from the equation
\overline{J}=−k∇Φ;
(e) comparing the voltages calculated in step (c) for each of said selected voltage measuring sites of said structure and the corresponding voltages measured at said selected sites in step (b);
(f) repeating steps (c) and (d) when the difference between the voltages compared in step (3) are greater than a predetermined amount, the voltages measured in step (b) then being substituted at said selected voltage measuring sites for the voltages calculated in step (c);
(g) calculating new values for k for each of said locations when the squared residual sum R equals
$$\frac{\sum }{X} \int \int v ( \overline{J} j + k ∇ φ ) \cdot ( \overline{J} + k ∇ φ ) ⅆ v$$
Where V is the region over which the imaging is being performed and X represents the excitations over which the sum is taken, by determining the values of K which minimize R throughout said structure; and
(h) repeating steps (f) and (g) until the voltages compared in step (e) do not exceed said predetermined amount.

11. A method of detecting malignant and benign tumors in a breast comprising positioning an electrode array on the surface of the breast, passing current between selected pairs of electrodes sequentially, measuring the voltages between electrode pairs not carrying said currents, and calculating the position, size and malignancy of tumors from the potential and conductivity information derived from said voltage measurements.

12. A method of detecting malignant and benign tumors in a breast comprising positioning an electrode array on the inner surface of a dielectric container containing a conductive fluid, immersing said breast in said fluid, passing current between selected pairs of electrodes sequentially, measuring the voltages between electrode pairs not carrying said currents, and calculating the position, size, and malignancy of tumors from the potential and conductivity information derived from said voltage measurements.

13. A method of detecting malignant and benign tumors in a body part comprising positioning an electrode array on the surface of the body part, passing current between selected pairs of electrodes sequentially, measuring the voltages between electrode pairs not carrying said currents, and calculating the position, size and malignancy of tumors from the potential and conductivity information derived from said voltage measurements.

14. A method of detecting malignant and benign tumors in a body part comprising positioning an electrode array on the inner surface of a dielectric container containing a conductive fluid, immersing said body part in said fluid, passing current between selected pairs of electrodes sequentially, measuring the voltages between electrode pairs not carrying said currents, and calculating the position, size, and malignancy of tumors from the potential and conductivity information derived from said voltage measurements.

15. A method as in claim 5 and displaying the calculated position, size and malignancy.

16. A method as in claim 6, and displaying the calculated position, size and malignancy.
